(19) United States
(12) Patent Application Publication
(10) Pub. No.: US 2026/0027281 A1
(43) Pub. Date: Jan. 29, 2026

(54) MANAGING PERIPHERAL BLOOD CELLS

(71) Applicant: SINO-BIOCAN (SHANGHAI) BIOTECH LTD., Shanghai (CN)

(72) Inventor: Dongbing WEI, Shanghai (CN)

(21) Appl. No.: 18/997,637

(22) PCT Filed: Aug. 8, 2023
(86) PCT No.: PCT/CN2023/111658

§ 371 Date: Jan. 22, 2025

(30) Foreign Application Priority Data
Aug. 9, 2022 (CN) 202222086487.2
Dec. 21, 2022 (CN) 202211647865.8

(51) Int. Cl. A61M 1/36; B04B 5/04; B04B 9/10; B04B 11/04

(52) U.S. Cl. CPC A61M 1/3695; B04B 5/0428; B04B 9/10; B04B 11/04

(57) ABSTRACT

Methods, devices, and systems for managing peripheral blood cells are provided. In one aspect, a fully automatic peripheral blood separation method includes: adding a peripheral blood sample liquid in a sample bag and a gradient liquid into a centrifuge cup, collecting a target cell liquid into an intermediate bag after centrifugation of the peripheral blood sample liquid and the gradient liquid in the centrifuge cup, adding the target cell liquid in the intermediate bag and a washing liquid to the centrifuge cup for washing and replacement to obtain a pre-product, and mixing the pre-product and a cell dilution liquid to obtain a final product.

### CROSS REFERENCE TO RELATED APPLICATIONS

This application claims priority to Chinese Patent Application No. 202222086487.2, filed on Aug. 9, 2022, and Chinese Patent Application No. 202211647865.8, filed on Dec. 21, 2022, the entire contents of which are incorporated herein by reference.

### TECHNICAL FIELD

The present disclosure relates generally to the field of biotechnology, in particular to methods, devices, and systems for managing peripheral blood cells.

### BACKGROUND

Cell therapy refers to utilizing the characteristics of particular cells with specific functions, employing bioengineering methods, e.g., in vitro expansion, special culture and other types of treatments, to produce cells that can enhance immunity, kill pathogens and tumor cells, promote tissue and organ regeneration and body recovery, provide other therapeutic effects, and then using the cells to treat various diseases. However, separating and packaging peripheral blood cells are challenging.

### SUMMARY

The present disclosure describes methods, devices, and systems for managing peripheral blood cells, e.g., separating and packaging peripheral blood mononuclear cells (PBMCs).

One aspect of the present disclosure provides a fully automatic peripheral blood separation method. The method includes: adding a peripheral blood sample liquid in a sample bag and a gradient liquid into a centrifuge cup; collecting a target cell liquid into an intermediate bag after centrifugation of the peripheral blood sample liquid and the gradient liquid in the centrifuge cup; adding the target cell liquid in the intermediate bag and a washing liquid to the centrifuge cup for washing and replacement to obtain a pre-product; and mixing the pre-product and a cell dilution liquid to obtain a final product.

In some implementations, the method further includes one or more steps of pre-separation treatment, setting separation parameters, starting separation, and ending the process. The method can be performed at least partially by a fully automatic peripheral blood separation device.

In some implementations, the pre-separation treatment step includes: installing or adding one or more consumables on the fully automatic peripheral blood separation device, and then connecting a peripheral blood sample liquid (e.g., peripheral blood), a gradient liquid, a washing liquid (e.g., replacement liquid), and a cell dilution liquid.

In some implementations, the peripheral blood sample liquid in the sample bag is firstly degassed, and then added to the centrifuge cup, and a liquid feeding rate at this time is about 18 to 22 mL/min. In some examples, the liquid feeding rate is 20 mL/min.

In some implementations, different layers are formed following the centrifugation, and then the sample liquid remaining in a pipe is returned to the sample bag.

In some implementations, when a volume of the peripheral blood sample liquid is greater than a separation volume of the centrifuge cup, a plurality of times of feeding and separation are performed for the sample liquid.

In some implementations, after collecting the target cell liquid, the centrifuge cup and the pipe are cleaned.

In some implementations, an order for adding the target cell liquid and the washing liquid into the centrifuge cup is: firstly discharging the target cell liquid from the intermediate bag to the centrifuge cup, then adding the washing liquid to the intermediate bag, and then discharging the washing liquid to the centrifuge cup.

In some implementations, the washing and replacement are carried out for 1 to 3 times. In some examples, the washing and replacement are carried out for 3 times.

In some implementations, after the final product is obtained, the final product is discharged into a product bag, the centrifuge cup is cleaned with the cell dilution liquid, and the cleaned liquid is discharged into the product bag, and the centrifuge cup is cleaned for 1 to 20 times. In some examples, the centrifuge cup is cleaned for 3 times.

In some implementations, after cleaning the centrifuge cup, the final product in the product bag is packaged. In some implementations, after packaging the final product in the product bag, residual collection is carried out.

Another aspect of the present disclosure features a fully automatic peripheral blood separation device, and the fully automatic peripheral blood separation method described above is applied to the device.

In some implementations, the fully automatic peripheral blood separation device includes a sample scale, a replacement liquid 2 scale, a replacement liquid 1 scale, a centrifuge, an intermediate scale, a product scale, a touch screen, an emergency stop button, a bubble detector, a centrifuge pressure monitor, a peristaltic pump, a spin valve, and an alarm system.

The sample scale is configured to weigh the feeding amount of the peripheral blood sample liquid. The replacement liquid 1 scale and the replacement liquid 2 scale are configured to weigh the feeding amount of the replacement liquid (washing liquid). The centrifuge is configured to provide a rotational power to the centrifuge cup to separate the cell liquid. The intermediate scale is configured to weigh the target cell liquid in the intermediate bag. The product scale is configured to weigh the finished product. The touch screen is configured to set the separation parameters. The emergency stop button is configured to stop the rotation of the centrifuge. The bubble detector is configured to detect whether there are bubbles in the pipe. The centrifuge pressure monitor is configured to monitor the pressure inside the centrifuge to ensure the safety of the centrifuge process. If the pressure of the centrifuge is too high, the alarm system issues an alarm signal, and the emergency stop button is immediately pressed to stop the operation of the centrifuge. The peristaltic pump is configured to pump a liquid into or out of a centrifuge cup. The spin valve is configured to control the opening and closing of the pipe.

Another aspect of the present disclosure features a fully automatic peripheral blood separation device, including: a plurality of components and a controller coupled to one or more components of the plurality of components. The plurality of components includes at least one of: a centrifuge configured to provide a rotational power to a centrifuge cup, a spin valve configured to control opening or closing of a pipe coupled to the centrifuge cup, a peristaltic pump configured to pump a liquid into or out of the centrifuge cup, or one or more scales each configured to weigh an amount of a respective liquid. The controller is configured to perform one or more operations for fully automatic peripheral blood separation. The one or more operations include: controlling the spin valve to open the pipe and the peristaltic pump to pump a peripheral blood sample liquid and a gradient liquid into the centrifuge cup; controlling the centrifuge to rotate to separate a target cell liquid from the peripheral blood sample liquid and the gradient liquid in the centrifuge cup; controlling to collect the target cell liquid into an intermediate bag; controlling to add the target cell liquid in the intermediate bag and a washing liquid to the centrifuge cup for washing and replacement to obtain a pre-product; and controlling to mix the pre-product and a cell dilution liquid to obtain a final product.

Another aspect of the present disclosure features a PBMC separating and packaging device. The device includes: a washing liquid assembly, a density gradient liquid assembly, a sample liquid assembly, a waste liquid assembly, a preparation liquid assembly, a semi-finished product liquid assembly, a packaging assembly, and a separation cup. The separation cup is connected to a first three-way joint via a cup inlet pipe, and the first three-way joint is further connected to a liquid inlet pipe and a collection pipe. The washing liquid assembly, the density gradient liquid assembly, the sample liquid assembly, and the waste liquid assembly are connected to the liquid inlet pipe via a first five-way joint. The preparation liquid assembly, the semi-finished product liquid assembly and the packaging assembly are connected to the collection pipe via a first four-way joint.

In some implementations, the washing liquid assembly includes a first washing liquid branch, a second washing liquid branch, and a washing liquid pipe. One end of the washing liquid pipe is connected to the first five-way joint, and the other end of the washing liquid pipe is connected to the first washing liquid branch and the second washing liquid branch via a second three-way joint.

In some implementations, a protective cap, a first piercer, a first Luer connector and a first pipe clamp are provided on the first washing liquid branch, and a second piercer and a second pipe clamp are provided on the second washing liquid branch.

In some implementations, the density gradient liquid assembly includes a density gradient liquid pipe and a density gradient liquid bag; one end of the density gradient liquid pipe is connected to the first five-way joint, the other end of the density gradient liquid pipe is connected to the density gradient liquid bag; a third pipe clamp is provided on the density gradient liquid pipe; and a liquid filter and a fourth pipe clamp are provided on the density gradient liquid bag.

In some implementations, the sample liquid assembly includes a sample pipe. The sample pipe can be connected to the first five-way joint, and a third piercer, a second Luer connector, a connecting pipe, a blood filter and a fourth pipe clamp can be provided on the sample pipe.

In some implementations, the waste liquid assembly includes a waste liquid pipe. One end of the waste liquid pipe can be connected to the first five-way joint, and the other end of the waste liquid pipe is connected to a waste liquid bag. A fifth pipe clamp can be provided on the waste liquid pipe.

In some implementations, the preparation liquid assembly includes a first preparation liquid branch, a second preparation liquid branch, and a preparation liquid pipe. One end of the preparation liquid pipe is connected to the first four-way joint, the other end of the preparation liquid pipe is connected to the first preparation liquid branch and the second preparation liquid branch via a fourth three-way joint. A seventh pipe clamp and a fourth piercer can be provided on the first preparation liquid branch. An eighth pipe clamp and a preparation liquid bag can be provided on the second preparation liquid branch. The preparation liquid bag can be provided with a third Luer connector.

In some implementations, the semi-finished product liquid assembly includes a semi-finished product bag pipe. One end of the semi-finished product bag pipe is connected to the first four-way joint, and the other end of the semi-finished product bag pipe is connected to the semi-finished product bag. A ninth pipe clamp can be provided on the semi-finished product bag pipe.

In some implementations, the packaging assembly includes a packaging pipe. One end of the packaging pipe is connected to the first four-way joint, and the other end of the packaging pipe is connected to a cryopreservation container. A tenth pipe clip can be provided on the packaging pipe.

In some implementations, the separation cup is connected to the cup inlet pipe and a pressure monitoring pipe via a third three-way joint. A sixth pipe clamp and a pressure monitoring joint can be provided on the pressure monitoring pipe.

The technical solutions in the embodiments of the present disclosure will be clearly and fully described below in conjunction with the accompanying drawings for these embodiments of the present disclosure. Apparently, the described embodiments are only some of the embodiments of the present disclosure, but not all of them. Based on these embodiments of the present disclosure, all other embodiments obtained by a person of ordinary skill in the art without creative efforts should fall within the scope of protection of the present disclosure.

The details of one or more embodiments of the present disclosure are set forth in the accompanying drawings and the description below. Other embodiments and advantages of the present disclosure will be apparent from the description and drawings, and from the claims.

### BRIEF DESCRIPTION OF THE DRAWINGS

FIG. 1A illustrates a perspective view of an example fully automatic peripheral blood separation device.

FIG. 1B illustrates a side view of the fully automatic peripheral blood separation device of FIG. 1A.

FIG. 2 is a flow chart of an example process of a fully automated peripheral blood separation method.

FIG. 3 is a flow chart of an example process of initiating separation and packaging.

FIG. 4 is a flow chart of an example process of gradient liquid separation.

FIG. 5 is a schematic diagram of an example PBMC separating and packaging device.

Like reference numbers and designations in the various drawings indicate like elements. It is also to be understood that the various exemplary implementations shown in the figures are merely illustrative representations and are not necessarily drawn to scale.

### DETAILED DESCRIPTION

Peripheral blood mononuclear cell (PBMC) is a type of cells with a single nucleus in peripheral blood, including lymphocytes and monocytes. Methods for separating peripheral blood mononuclear cells includes adhesion method, immunomagnetic bead separation method, flow cytometry separation method, and the Ficoll-Hypaque density gradient centrifugation method. The adhesion method is cumbersome to operate, the recovery rate is low, and the separation purity is not high. The costs of the immunomagnetic bead method and flow cytometry separation are both high. For the Ficoll-Hypaque density gradient centrifugation method, the sample size cannot be too large. If the sample size is too large, it may affect the separation effect. In addition, this method has relatively high requirements on operators, poor consistency of products, and low production efficiency. Hydroxyethyl starch precipitation and Ficoll-Hypaque density gradient centrifugation can be combined to achieve further purification of mononuclear cells. However, its operation is manual operation, the production rate is low, and the product consistency is also poor. In addition, it still suffers from the problem of poor recovery rate.

A separating and packaging process of PBMC can include: separating peripheral blood or umbilical cord blood, washing to obtain PBMC, manually adding a cryopreservation liquid to the PBMC in a grade A clean environment, and then manually packaging into cryopreservation pipes or cryopreservation bags in small volumes (e.g., 1 mL, 2 mL, or 5 mL) for storage. The packaging process can include: firstly sealing the PBMC with a cryopreservation liquid with a heat scaler, and then connecting a cryopreservation pipe or a cryopreservation bag with a sterile tubulating machine; after the cryopreservation pipe or cryopreservation bag is full, connecting the next cryopreservation pipe or cryopreservation bag with the sterile tubulating machine, and repeating the foregoing process until the packaging is completed. However, the above-mentioned packaging process is cumbersome and redundant, and there is an open environment in the manual operation mode, which is prone to contamination.

Implementations of the present disclosure provide methods, device, systems, and techniques for managing peripheral blood cells, e.g., separation and packaging, which can solve the above-mentioned technical problems and cure the deficiencies of existing peripheral blood cells separation and/or packaging methods and devices. The techniques enable to automatically complete the separating and packaging of peripheral blood cells (e.g., PBMC) under a closed condition, which can reduce/eliminate contamination, be easier to operate, reduce human operation errors, improve production rate and production consistency, improve recovery rate and product purity, reduce sample loss, and realize compact configurations.

In some implementations, the techniques provide methods and devices for fully automatic peripheral blood separation and/or packaging. The methods can implement an automatic operation, which can integrate providing disposable consumables, separation, washing and replacement, and preparation packaging on a single device, e.g., a fully automatic peripheral blood separation device. The techniques can not only reduce work intensity of an operator, lower requirements on the operator, but also improves a production rate and product consistency.

In some implementations, using the fully automatic peripheral blood separation method, the loss of the sample liquid is reduced, and the recovery rate is improved by degassing the gradient liquid, slowly adding the sample liquid, and backflowing the sample liquid. In addition, the step of discharging the concentrated waste liquid can reduce red blood cells mixed in the product and improve the purity of the product. In the residual washing step for the centrifuge cup, the residual red blood cells in the pipe and the centrifuge cup are removed, which can further improve the product purity. The product can include PBMC cells. The techniques enable to load a large amount of sample liquid at one time, which can overcome the disadvantage of loading sample fluid for too many times in the Ficoll-Hypaque density gradient centrifugation method. Moreover, the techniques use disposable fully sealed consumables, which can reduce or eliminate the risk of contamination.

In some implementations, the techniques provide a PBMC separating and packaging device, which can be configured to separate PBMC cells from a peripheral blood sample or umbilical cord blood sample, e.g., the product obtained using the fully automatic peripheral blood separation methods and/or devices described in the present disclosure. In the PBMC separating and packaging device, the pipes are reasonably connected with corresponding multi-channel joints. The PBMC separating and packaging device can realize separation, washing, preparation, packaging and other functions for PBMC cells under a closed condition at one time. The PBMC separating and packaging device can reduce the number of open procedures, reduce the need to repeatedly connecting pipes for preparation, can package the product into cryopreservation samples of different specifications and volumes, can directly freeze the product for storage, can be easy to operate, and can ensure the safety of PBMC cells. Besides the PBMC cells, the techniques implemented in the present disclosure can be also applied to any other suitable cells.

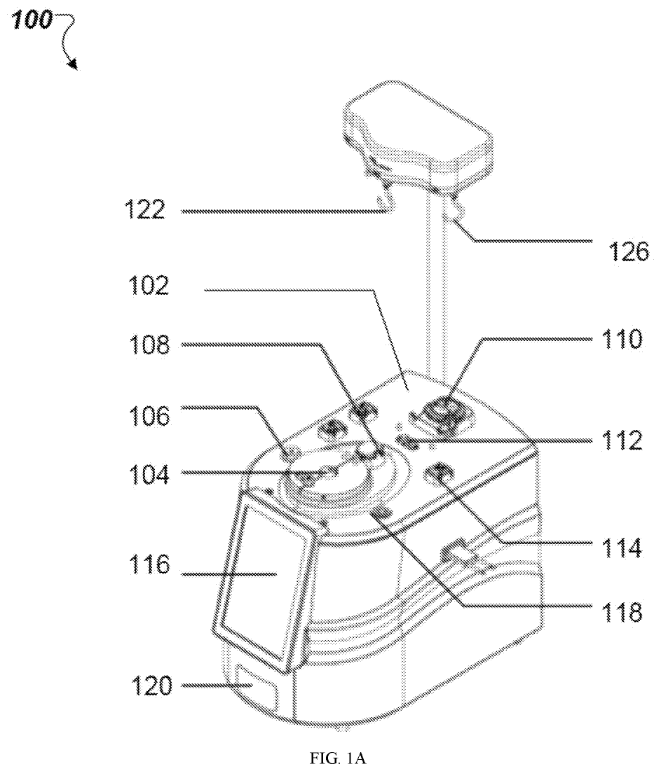

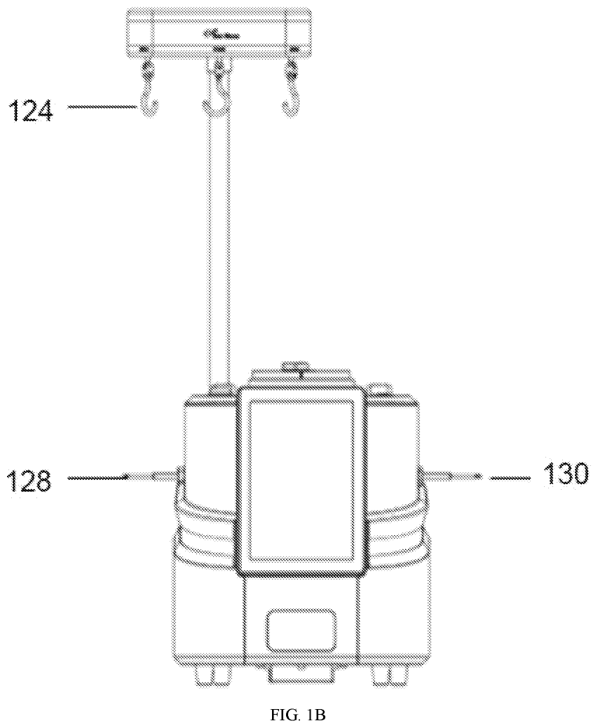

FIGS. 1A-1B illustrate a perspective view of an example fully automatic peripheral blood separation device 100. The fully automatic peripheral blood separation device 100 includes a device body 102 and a number of components that can be assembled on the device body 102. In some implementations, the fully automatic peripheral blood separation device 100 includes a centrifuge 104, a centrifuge pressure monitor 106, a pipe detector 108, a peristaltic pump 110, a bubble detector 112, a spin valve 114, a touch screen 116, an emergency stop button 118, an alarm system 120, and a number of scales including a sample scale 122, a replacement liquid 1 scale 124, a replacement liquid 2 scale 126, an intermediate scale 128, and a product scale 130.

Functions of components of the fully automatic peripheral blood separation device 100 can be described as follows. In some implementations, the sample scale 122 is configured to weigh a feeding amount of a peripheral blood sample liquid. The replacement liquid 1 scale 124 and the replacement liquid 2 scale 126 can be configured to weigh a feeding amount of a replacement liquid (or washing liquid). The centrifuge 104 can be configured to provide a rotational power to a centrifuge cup to separate the cell liquid. The intermediate scale 128 can be configured to weigh a target cell liquid in an intermediate bag. The product scale 130 can be configured to weigh a finished product. The touch screen 116 can be configured to set one or more separation parameters. The emergency stop button 118 can be configured to stop the rotation of the centrifuge 104. The bubble detector 112 can be configured to detect whether there are bubbles in a pipe. The pipe can connect with different components, including the centrifuge cup and one or more reservoirs including a sample bag storing a peripheral blood sample liquid, an intermediate bag for storing a target cell liquid, and/or a product bag storing the finished product. The pipe detector 108 can be configured to detect whether there is any residual liquid in the pipe. The centrifuge pressure monitor 106 can be configured to monitor the pressure inside the centrifuge 104 to ensure the safety of the centrifuge process. If the pressure of the centrifuge 104 is too high, the alarm system 120 can issue an alarm signal, e.g., sound and/or light and/or vibration. The emergency stop button 118 can be immediately pressed to stop the operation of the centrifuge 104. The peristaltic pump 110 can be configured to pump a liquid into or out of the centrifuge cup. The spin valve 114 can be configured to control the opening and closing of the pipe.

The fully automatic peripheral blood separation device 100 can include a controller. The controller can include at least one processor and at least one memory storing instructions that are executable by the at least one processor to perform one or more operations as described in the present disclosure. In some implementations, the controller is coupled to one or more components of the fully automatic peripheral blood separation device 100. The one or more components can include the centrifuge 104, the centrifuge pressure monitor 106, the pipe detector 108, the peristaltic pump 110, the bubble detector 112, the spin valve 114, the touch screen 116, the emergency stop button 118, the alarm system 120, and/or one or more of the sample scale 122, the replacement liquid 1 scale 124, the replacement liquid 2 scale 126, the intermediate scale 128, and the product scale 130. The controller can communicate with the one or more components, receive signals from the one or more components, and/or send signals to control operation of the one or more components. For example, the controller can receive a signal indicating the pressure inside the centrifuge 104 from the centrifuge pressure monitor 106. If the controller determines, based on the signal, that the pressure of the centrifuge 104 is higher than a predetermined pressure threshold, the controller can transmit a control signal to the alarm system 120 to generate an alarm signal and/or a control signal to the centrifuge 104 to reduce a rotation speed/power of the centrifuge 104 or to stop the rotation of the centrifuge 104.

In some implementations, the controller is configured to control the centrifuge 104 to rotate to provide a rotational power to a centrifuge cup to separate the cell liquid. The controller can also be configured to receive one or more separation parameters from the touch screen 116 and control one or more components (e.g., the centrifuge 104) based on the one or more separation parameters. The controller can receive a detection signal from the bubble detector 112, the signal indicating whether there are bubbles in the pipe. If the controller determines, based on the detection signal, that there are bubbles in the pipe, the controller can control the spin valve 114 to open the pipe to get rid of the bubbles from the pipe. The controller can also control the peristaltic pump 110 pump a liquid into or out of the centrifuge cup.

In some implementations, the controller is configured to receive weight values from the sample scale 122, the replacement liquid 1 scale 124, the replacement liquid 2 scale 126, the intermediate scale 128, and/or the product scale 130, and take corresponding actions based on the weight values. For example, if the weight of the target cell liquid in the intermediate bag is lower than a predetermined threshold, the controller can control the peristaltic pump 110 to pump more liquid into the centrifuge cup. As another example, if the weight of the finished product is lower than a predetermined weight, the controller can control the alarm system 120 to generate an alarm signal or control the centrifuge 104 to continue rotating to obtain more target cell liquid.

In some implementations, the fully automatic peripheral blood separation device 100 is configured to perform a fully automatic peripheral blood separation method as discussed in the present disclosure. The method can include: adding a peripheral blood sample liquid (e.g., stored in a sample bag) and a gradient liquid into a centrifuge cup, collecting a target cell liquid into an intermediate bag after centrifugation (e.g., by the centrifuge 104), adding the target cell liquid (e.g., collected in the intermediate bag) and a washing liquid to a centrifuge cup for washing and replacement to obtain a pre-product, and mixing the pre-product and a cell dilution liquid to obtain a final product.

Different layers can be formed following the centrifugation, and then the sample liquid remaining in a pipe can be returned to the sample bag. When a volume of the peripheral blood sample liquid is greater than a separation volume of the centrifuge cup, the sample liquid can be fed and separated for multiple times. After collecting the target cell liquid, the centrifuge cup and the pipe can be cleaned.

In some implementations, an order for adding the target cell liquid and the washing liquid into the centrifuge cup is: first discharging the target cell liquid from the intermediate bag to the centrifuge cup, then adding the washing liquid to the intermediate bag, and then discharging the washing liquid to the centrifuge cup. After the final product is obtained, the final product can be discharged into a product bag. The centrifuge cup can be cleaned with the cell dilution liquid, and the cleaned liquid can be discharged into the product bag. After cleaning the centrifuge cup, the final product in the product bag can be packaged, and then residual collection can be carried out.

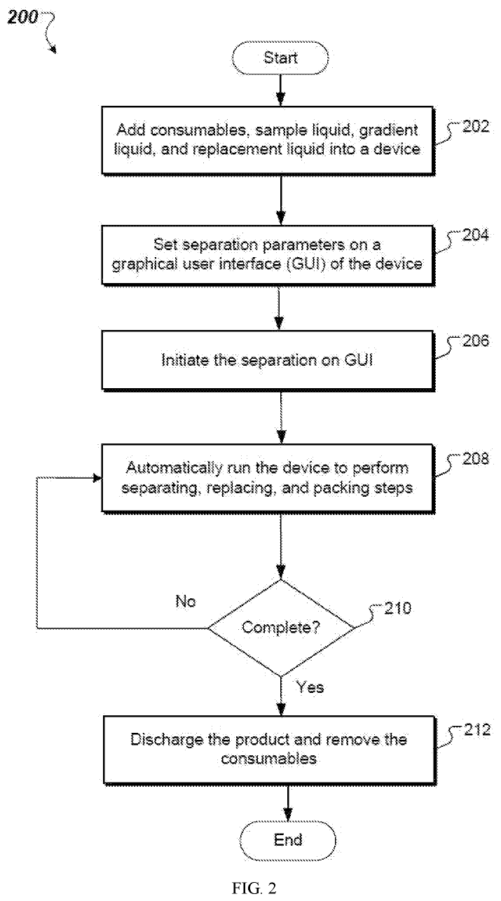

FIG. 2 is a flow chart of an example process 200 of a fully automated peripheral blood separation method. The process 200 can be at least partially performed by the fully automatic peripheral blood separation device 100 of FIGS. 1A-1B. For simplicity, the fully automatic peripheral blood separation device 100 can be referred to as the device 100. The process 200 can include one or more steps including: pre-separation treatment (e.g., step 202), setting separation parameters (e.g., step 204), starting separation (e.g., step 206), automatic separation (e.g., steps 208, 210), product discharge and consumables removal (e.g., step 212), and ending the process.

The pre-separation treatment step 202 can include: adding one or more consumables (e.g., PBMC consumables) on the device 100, and then separately connecting the one or more consumables to individual reservoirs/bags storing a peripheral blood sample liquid (which can be referred to peripheral blood or sample fluid), a gradient liquid, a washing liquid (or replacement liquid), and a cell dilution liquid.

At step 204, the separation parameters can be set with the touch screen 116 of the device 100, e.g., by a user input. In some implementations, the separation parameters arc predetermined and/or preconfigured as default values in the device 100. An operator can adjust the predetermined values through the touch screen 116. The separation parameters to be set can include: the time of the gradient liquid timing phase, the rate of sample feeding, the number of separation cycles, the determination of the end of the separation cycle, the repeating number for washing the residue in the centrifuge cup, and/or the repeating times for washing the centrifuge cup.

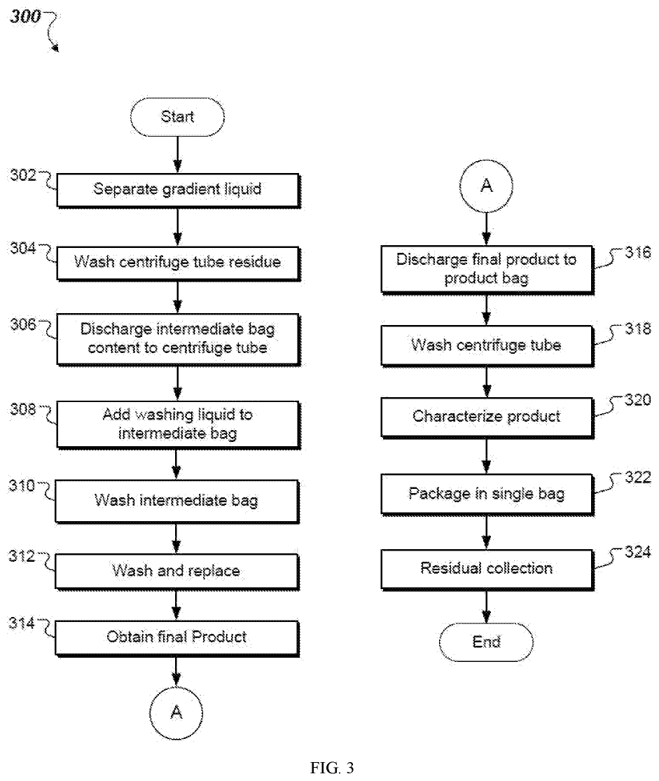

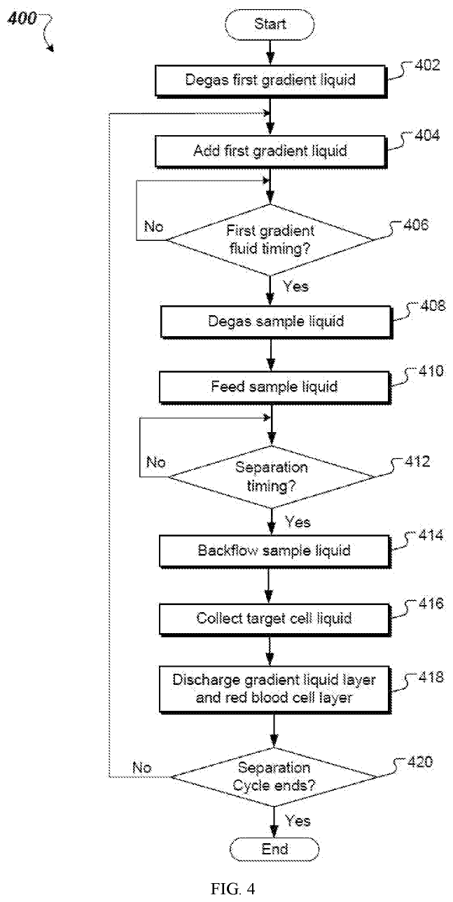

Subsequently, the separation process is initiated at step 206, e.g., by receiving a user input through a graphical user interface (GUI) on the touch screen 116. The device 100 can automatically run the steps of separating, replacing, and/or packaging at step 208, e.g., by performing the process 300 of FIG. 3 and/or process 400 of FIG. 4 as described with further details below. At step 210, the device 100 determines whether the separation process is completed. If the separation process is not completed, the process 200 returns to step 208. If the separation process is completed, the process 200 proceeds to step 212, where the product is discharged and the consumables are disposed to complete the entire process of the automatic separation method.

FIG. 3 is a flow chart of an example process 300 of initiating separation and packaging. The process 300 can be implemented as an example of the separation process of FIG. 2 (e.g., steps 208 and 210 of FIG. 2). The process 300 can be performed by the fully automatic peripheral blood separation device 100 (e.g., the controller). In some implementations, the process 300 includes multiple steps, e.g., from step 302 to step 324.

At step 302, gradient liquid is separated. FIG. 4 is a flow chart of an example process 400 of gradient liquid separation. The process 400 can be implemented as an example of the step 302 of FIG. 3. The process 400 can also be performed by the fully automatic peripheral blood separation device 100 (e.g., the controller).

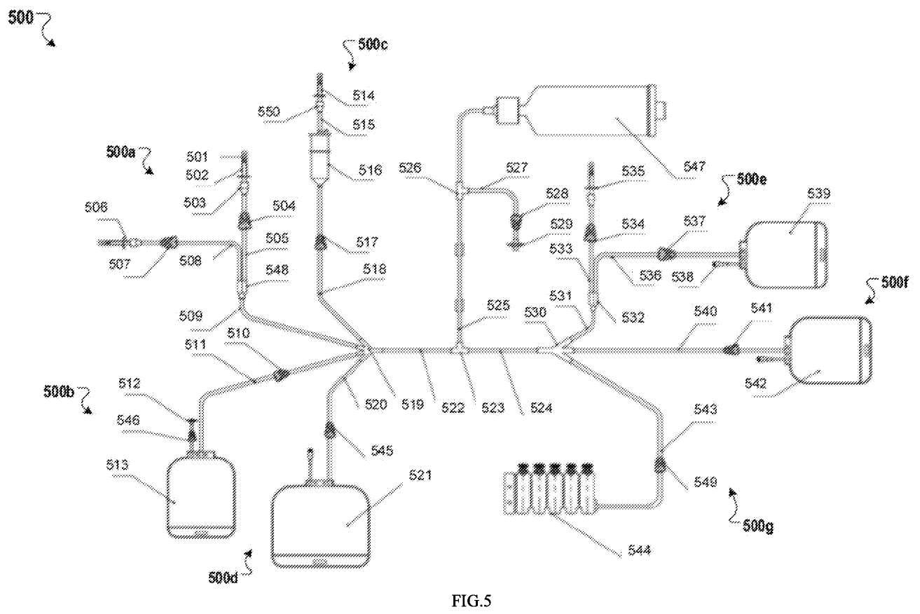

Referring to FIG. 4, the process 400 can include multiple steps. As air exists in an enclosed pipe, when the first gradient liquid flows into the centrifuge cup, the air in the pipe may also enter into the centrifuge cup. Performing step 402 can get rid of the air from the centrifuge cup, which can make sure that the pipe and the centrifuge cup are full of liquid and can guarantee the stability of the centrifuge process. At step 402, the device 100 degases the first gradient liquid and fills the first gradient liquid in the pipe and the centrifuge cup. Step 402 can include three substeps: 1) the device 100 can open the spin valve 114 to add the first gradient liquid from a sample bag to the centrifuge cup (e.g., a separation cup 547 of FIG. 5); 2) the device 100 can remove the air from an upper part of the centrifuge cup to a waste liquid bag, which can make sure that the pipe is full of the first gradient fluid, 3) the device 100 can flow the first gradient liquid again from the sample bag through the pipe to the centrifuge cup. As performing substeps 1) and 2) can remove the air completely from the pipe and the centrifuge cup. As the pipe is filled up with the first gradient liquid, there can be no more air in the centrifuge cup.

At step 404, the device 100 adds the degassed first gradient liquid to the centrifuge cup to obtain a centrifuge cup filled with the first gradient liquid. At step 406, the device 100 turns on the centrifuge 104, and sets the centrifugation time to a predetermined time period (e.g., 30 seconds). The device 100 can determine whether the centrifugation time is over. If the centrifugation time is not over, the device 100 continues rotating the centrifuge 104. If the centrifugation time is over, the device 100 adds the blood sample to the centrifuge cup, while keeping the centrifuge 104 running. When all blood samples are added into the centrifuge cup, the centrifuge 104 keeps running for a while until an interface of different layers appears in the centrifuge cup. When the centrifugation time is over, during the condition of centrifuging, the device 100 starts the moving components to move up the pistol inside the centrifuge cup and expel out the liquid in the centrifuge cup into the pipe. In some implementations, the device 100 includes a monitoring system (e.g., an optical detection system such as the pipe detector 108 of FIG. 1) configured to detect components of the liquid in the pipe. By controlling the spin valve 114 of the device 100, the device 100 can collect the sample liquid into a sample bag. After completing the collection of the sample liquid, the device 100 (e.g., the controller) can control the centrifuge 104 to stop, and expel out the remaining gradient fluid and red blood cells into the waste liquid bag.

At step 408, the device 100 degasses the peripheral blood sample liquid (or sample fluid). The device 100 can first weigh a feeding volume of the sample liquid in a sample bag with the sample scale 122, and add the sample liquid contained in the sample bag to the centrifuge cup. At this time, the centrifuge cup piston can be controlled to move up to discharge the air in the upper part of the centrifuge cup and the pipe, and at the same time, the device 100 can discharge the sample liquid, so as to finish the degassing process. Subsequently, at step 410, the device 100 can transfer the sample fluid from the sample bag into the centrifuge cup after the degassing process at step 408 is finished.

The device 100 can start the centrifuge 104 so that the centrifuge cup showing the interface of different layers in step 406 is in a rotating state. At this time, the centrifuge cup is filled with the gradient liquid. Subsequently, at step 410, the sample liquid is added to the centrifuge cup at a predetermined rate (e.g., a rate of 18 to 22 mL/min) until the sample addition is finished. In some examples, the rate is 20 mL min.

At step 412, the centrifuge 104 can be set to a centrifugation timing stage via the touch screen 116 of the device 100, or by a predetermined setting for the centrifuge 104 in the device 100. The device 100 determines whether a predetermined time period (e.g., 25 minutes) for the centrifugation timing stage is over. If the predetermined time period is not over, the centrifuge 104 keeps rotating. If the predetermined time period is over, the device 100 stops the centrifuge 104. The predetermined time period can be configured such that different layers are shown in the centrifuge cup. From top to bottom in the centrifuge cup, there can be a plasma layer, a target cell liquid layer, a gradient liquid layer, and a red blood cell layer.

At step 414, the device 100 backflows the sample liquid from the centrifuge cup that has different layers. At this time, the device 100 can detect the residual peripheral blood sample liquid in the pipe with the pipe detector 108, and then transfer the top plasma layer to the pipe, and return part of the plasma layer and the residual sample liquid in the pipe to the sample bag. After repeating step 412, the device 100 can transfer the plasma layer from the centrifuge cup to the waste liquid bag.

The centrifuge cup that has drained the plasma layer in step 414 still has the target cell liquid, the gradient liquid layer, and the red blood cell layer. At step 416, the device 100 performs cell liquid collection. The device 100 can open the spin valve 114 to discharge the target cell liquid from the centrifuge cup to the intermediate bag, and at the same time, weigh the target cell liquid with the intermediate scale 128.

At step 418, the device 100 discharges the gradient liquid layer and red blood cell layer. After collecting the target cell liquid at step 416, the device 100 stops the centrifuge cup, and then discharge the gradient liquid layer and the red blood cell layer to the waste liquid bag.

At step 420, the device 100 determines whether the separation cycle ends. If the amount of the peripheral blood sample liquid is greater than the separation volume of the centrifuge cup, the peripheral blood sample liquid can be divided for a plurality of times of feeding and separation. The repeating number can be determined according to the total amount of the sample liquid and the separation volume of the centrifuge cup. Thus, the separation cycle includes the plurality of times for repeating steps 404 to 420. If the repeating number is less than the plurality of times, the process 400 returns to step 404 and continues. If the separation cycle ends, the process 400 ends.

Referring back to FIG. 3, after the gradient fluid is separated at step 302 or by the process 400, the device 100 washes centrifuge cup residue at step 304. In some implementations, the washing liquid is added to the centrifuge cup to clean the remaining red blood cells from the centrifuge cup and the pipe, and then discharge the remaining red blood cells to the waste liquid bag. Step 304 can be repeated for a plurality of times, e.g., 3 times.

At step 306, the intermediate bag content is discharged to the centrifuge cup. The device 100 can open the spin valve 114 and discharge all the target cell liquid in the intermediate bag to the centrifuge cup following step 304. At this time, the intermediate bag still has residual target cell liquid.

At step 308, the washing liquid is added to the intermediate bag. Subsequently, the washing liquid is added to the intermediate bag containing the residual target cell liquid following step 306 for washing.

At step 310, the intermediate bag is washed. The device 100 can pump the washing liquid added to the intermediate bag in step 308 and the residual target cell liquid into the centrifuge cup obtained in step 306 by the peristaltic pump 110. After diluting and washing the cell liquid in the centrifuge cup, the device 100 can centrifuge the cell liquid, and then discharge the waste liquid to the waste liquid bag, and retain the cell liquid in the centrifuge cup.

At step 312, the device 100 performs washing and replacing. First, the device 100 can weigh a first washing liquid by the replacement liquid 1 scale 124, then pump the first washing liquid into the centrifuge cup obtained in step 310 with the peristaltic pump 110, and dilute and wash the cell liquid in the centrifuge cup with the first washing liquid. After centrifugation, the device 100 discharges the supernatant waste liquid to the waste liquid bag, and retains the cell liquid. Next the device 100 can weigh a second washing liquid with the replacement liquid 2 scale 126, and continue to add the second washing liquid to dilute and wash the cell liquid in the centrifuge cup. After centrifugation, the device 100 can discharge the supernatant waste liquid to the waste liquid bag and retain the cell liquid; continue to add a third washing liquid to dilute and wash the cell liquid in the centrifuge cup, after centrifugation, discharge the supernatant waste liquid to the waste liquid bag to obtain a pre-product. A specific number of repeating the washing and replacing steps can be, for example, 1 to 3 times. More washing and replacing steps can be carried out according to actual needs, which is not limited herein. The first washing liquid, the second washing liquid, and the third washing liquid can be the same or different, and can be selected according to actual needs.

At step 314, the cell dilution liquid is added to the centrifuge cup containing the pre-product obtained in step 312 to dilute the pre-product, so as to obtain the final product. At step 316, the device 100 discharges the final product into a product bag. The final product obtained in step 314 is discharged into a product bag, and meanwhile, the amount of the final product is weighed by the product scale 130.

At step 318, the centrifuge cup is cleaned. After the final product is discharged in step 316, a certain amount of residual final product may remain in the centrifuge cup. Thus, the cell dilution liquid can be pumped into the centrifuge cup to wash the residual final product remaining in the centrifuge cup and can be discharged into the product bag. The centrifuge cup can be cleaned for a number of times, e.g., 1 to 20 times. In some examples, the number of times is 3 times.

At step 320, the cell liquid discharged into the product bag in step 318 is sampled and counted. The number of recovered cells and the recovery rate can be calculated based on the volume, in combined with the sampled and counted number of cells in the cell liquid.

At step 322, the sampled cell liquid is packaged in one or more individual bags. After sampling, a preparation liquid is added based on the cell count information to dilute the cells to reach a packaging concentration, and then the final product is packaged into the one or more individual bags.

At step 324, residual is collected. After packaging and before removing the consumables, the final product remaining in the centrifuge cup is completely discharged by drawing air through a sterile filter of the consumables.

As noted above, the final product packaged in individual bags contains a target cell liquid including target cells, e.g., PBMC cells. The PBMC cells can be separated from the target cell liquid, e.g., by a PBMC separating and packaging device as discussed with respect to FIG. 5.

FIG. 5 is a schematic diagram of an example PBMC separating and packaging device 500. The PBMC separating and packaging device 500 can separate PBMC cells from a sample liquid, e.g., a peripheral blood sample or umbilical cord blood sample. The sample liquid can be, for example, the target cell liquid obtained by the fully automatic peripheral blood separation device 100 of FIGS. 1A-1B with the fully automated peripheral blood separation method as descried in FIGS. 2-4.

In some implementations, the PBMC separating and packaging device 500 includes a liquid inlet pipe 522, a collection pipe 524, a cup inlet pipe 525, a separation cup 547, and one or more assemblies, including a washing liquid assembly 500a, a density gradient liquid assembly 500b, a sample liquid assembly 500c, a waste liquid assembly 500d, a preparation liquid assembly 500e, a semi-finished product liquid assembly 500f, and a packaging assembly 500g.

In some implementations, e.g., as illustrated in FIG. 5, the liquid inlet pipe 522, the collection pipe 524, and the cup inlet pipe 525 are connected by a first three-way joint 523. The washing liquid assembly 500a, the density gradient liquid assembly 500b, the sample liquid assembly 500c, and the waste liquid assembly 500d can be connected to the liquid inlet pipe 522 via a first five-way joint 519. The preparation liquid assembly 500e, the semi-finished product liquid assembly 500f, and the packaging assembly 500g can be connected to the collection pipe 524 via a first four-way joint 530. The separation cup 547 can be connected to the cup inlet pipe 525.

In some implementations, the washing liquid assembly 500a includes a first washing liquid branch 505, a second washing liquid branch 508, and a washing liquid pipe 509. One end of the washing liquid pipe 509 is connected to the first five-way joint 519, and the other end of the washing liquid pipe 509 is connected to the first washing liquid branch 505, and the second washing liquid branch 508 via a second three-way joint 548. The first washing liquid branch 505 is provided with a protective cap 501, a first piercer 502, a first Luer connector 503, and a first pipe clamp 504. The second washing liquid branch 508 is provided with a second piercer 506 and a second pipe clamp 507.

In some implementations, the density gradient liquid assembly 500b includes a third pipe clamp 510, a density gradient liquid pipe 511, and a density gradient liquid bag 513. One end of the density gradient liquid pipe 511 is connected to the first five-way joint 519, and the other end of the density gradient liquid pipe 511 is connected to the density gradient liquid bag 513. The density gradient liquid bag 513 is provided with a liquid filter 512 and a fourth pipe clamp 546.

In some implementations, the sample liquid assembly 500c includes a sample pipe 518. One end of the sample pipe 518 is connected to the first five-way joint 519. The sample pipe 518 is provided with a third piercer 514, a second Luer connector 550, a connecting pipe 515, a blood filter 516, and a fourth pipe clamp 517.

In some implementations, the waste liquid assembly 500d includes a waste liquid pipe 520. One end of the waste liquid pipe 520 is connected to the first five-way joint 519, and the other end of the waste liquid pipe 520 is connected to the waste liquid bag 521. A fifth pipe clamp 545 can be provided on the waste liquid pipe 520.

In some implementations, the separation cup 547 is connected to the cup inlet pipe 525 and the pressure monitoring pipe 527 via a third three-way joint 526. The pressure monitoring pipe 527 can be provided with a sixth pipe clip 528 and a pressure monitoring joint 529. The pressure monitoring joint 529 can be used for monitoring the system pressure.

In some implementations, the preparation liquid assembly 500e includes a first preparation liquid branch 533, a second preparation liquid branch 536, and a preparation liquid pipe 531. One end of the preparation liquid pipe 531 is connected to the first four-way joint 530, and the other end of the preparation liquid pipe 531 is connected to the first preparation liquid branch 533 and the second preparation liquid branch 536 via a fourth three-way joint 532. A seventh pipe clamp 534 and a fourth piercer 535 can be arranged on the first preparation liquid branch 533. The second preparation liquid branch 536 can be provided with an eighth pipe clip 537 and a preparation liquid bag 539. The preparation liquid bag 539 can be provided with a third Luer connector 538.

In some implementations, the semi-finished product liquid assembly 500f includes a semi-finished product bag pipe 540. One end of the semi-finished product bag pipe 540 is connected to the first four-way joint 530, and the other end of the semi-finished product bag pipe 540) is connected to the semi-finished product bag 542. A ninth pipe clamp 541 can be provided on the semi-finished product bag pipe 540.

In some implementations, the packaging assembly 500g includes a packaging pipe 543. One end of the packaging pipe 543 is connected to the first four-way joint 530, and the other end of the packaging pipe 543 is connected to a cryopreservation container 544. A tenth pipe clamp 549 can be provided on the packaging pipe 543.

The working mechanism of the PBMC separating and packaging device 500 can be described as follows. In the PBMC separating and package device 500, the pipe clamps are configured to control on/off of respective pipes. The pipe clamps can be controlled by a controller. By using the pipe clamps to control the respective pipes to connect or disconnect, the PBMC separating and packaging device 500 can complete separation, washing, preparation, and packaging operations under a closed condition at one time. The control of the pipe clamps can be implemented in any suitable way.

In a separation process, a density gradient liquid is first injected into the density gradient liquid bag 513 via the liquid filter 512, the density gradient liquid then enters the separation cup 547 by way of the density gradient liquid pipe 511, the liquid inlet pipe 522 and the cup inlet pipe 525. Next, a peripheral blood or umbilical cord blood sample bag is connected by the third piercer 514 or the Luer connector 550. After being filtered by the blood filter 516, the blood sample enters the separation cup 547 via the sample pipe 518, the liquid inlet pipe 522, and the cup inlet pipe 525. The separation cup 547 can be driven by a power device to rotate at a high speed for separation. After the separation of the PBMC cells in the separation cup 547 is finished, the waste liquid produced in the separation enters the waste liquid bag 521 via the cup inlet pipe 525, the liquid inlet pipe 522 and the waste fluid pipe 520. The separated PBMC cells enter the semi-finished product bag 542 via the cup inlet pipe 525, the collection pipe 524 and the semi-finished product bag pipe 540.

In a washing process, the washing liquid is allowed to enter via the piercer 506 or the piercer 502, and then enter the separation cup 547 via the washing liquid pipe 509, the liquid inlet pipe 522, and the cup inlet pipe 525. Next the PBMC cells following the separation enter the separation cup 547 from the semi-finished product bag 542 via the semi-finished product bag pipe 540, the collection pipe 524, and the cup inlet pipe 525 to be mixed with the washing liquid for washing. After washing, the PBMC cells remain in the separation cup 547, while the generated waste liquid enters the waste liquid bag 521 via the cup inlet pipe 525, the liquid inlet pipe 522 and the waste fluid pipe 520.

In a preparation process, the preparation liquid (e.g., a cryopreservation liquid) is injected into the preparation liquid bag 539 by either the piercer 535 or the Luer connector 538. The preparation liquid can then enter the separation cup 547 via the preparation liquid pipe 531, the collection pipe 524, and the cup inlet pipe 525, and then the preparation liquid is mixed with the PBMC cells remaining in the separation cup 547.

In a packaging process, after mixed uniformly, the mixture enters the cryopreservation container 544 via the cup inlet pipe 525, the collection pipe 524, and the packaging pipe 543 to complete the packaging process of the PBMC cells. The packaged PBMC cell liquid can be stored with cryopreservation.

The transfer of liquid in the PBMC separating and packaging device 500 can be realized by one or more peristaltic pumps, e.g., the peristaltic pump 110 of FIGS. 1A-1B. The cryopreservation container 544 can be a multi-pack cryopreservation bag (e.g., a five-pack cryopreservation bag or a ten-pack cryopreservation bag) or a multi-pack cryopreservation tube (e.g., a five-pack cryopreservation tube or a ten-pack cryopreservation tube).

The disclosed and other examples can be implemented as one or more computer program products, for example, one or more modules of computer program instructions encoded on a computer readable medium for execution by, or to control the operation of, data processing apparatus. The computer readable medium can be a machine-readable storage device, a machine-readable storage substrate, a memory device, or a combination of one or more them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A system can encompass all apparatus, devices, and machines for processing data, including by way of example a programmable processor, a computer, or multiple processors or computers. A system can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a standalone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed for execution on one computer or on multiple computers that are located at one site or distributed across multiple sites and interconnected by a communications network.

The processes and logic flows described in this document can be performed by one or more programmable processors executing one or more computer programs to perform the functions described herein. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer can include a processor for performing instructions and one or more memory devices for storing instructions and data. Generally, a computer can also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data can include all forms of non-volatile memory, media and memory devices, including by way of example semiconductor memory devices, e.g., EPROM, EEPROM, and flash memory devices, magnetic disks. The processor and the memory can be supplemented by, or incorporated in, special purpose logic circuitry.

In the present disclosure, the terms “first” and “second” are only used for description, and cannot be understood as indicating or implying relative importance or implicitly indicating the number of the referred technical features. Thus, a feature defined as “first” and “second” may explicitly or implicitly include one or more of the features. The term “plurality” indicates two or more, unless otherwise specifically defined.

The terms “liquid” and “fluid” in the present disclosure can be used interchangeably. The terms such as “installing,” “connecting,” “associating” and “fixing” should be interpreted in a broad sense; for example, it can be fixedly connected, detachably connected, or integrally connected; it can be a mechanical connection or an electrical connection; it can be directly connected, or indirectly connected via an intermediary, and may be an internal communication between two elements or an interaction between two elements. A person of ordinary skill in the art can understand the specific meanings of the above terms according to specific situations.

In the present disclosure, unless otherwise explicitly specified or limited, that a first feature is “on” or “under” a second feature can be understood as that the first and second features are in direct contact, or the first and second features are in indirect contact via an intermediary. In addition, that a first feature is “on,” “over,” or “above” a second feature can be understood as that the first feature is directly above or obliquely above the second feature, or it may simply mean that the first feature is horizontally higher than the second feature. Moreover, that a first feature can be “under,” “below” or “underneath” a second feature can be understood as that the first feature is directly below or obliquely below the second feature, or it may simply mean that the first feature is horizontally lower than the second feature.

In the present disclosure, the terms “some implementations,” “one embodiment,” “some embodiments,” “example,” “specific examples,” or “some examples” refer to that the specific features, structures, materials or characteristics mentioned with the embodiment(s) or example(s) are included in at least one embodiment or example of the present disclosure. In this description, the schematic representations of the above terms are not necessarily directed to the same embodiment or example. Furthermore, the specific features, structures, materials or characteristics may be combined in any suitable manner in any one or more embodiments or examples. In addition, without conflict, a person skilled in the art can associate or combine different embodiments, examples, or features in different embodiments or examples described in this description.

While this document can describe many specifics, these should not be construed as limitations on the scope of the present disclosure that is claimed or of what can be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features can be described above as acting in certain combinations and even initially claimed as such, one or more features from a claimed combination in some cases can be excised from the combination, and the claimed combination can be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Only a few examples and implementations are disclosed. Variations, modifications, and enhancements to the described examples and implementations and other implementations can be made based on what is disclosed.

1. A fully automatic peripheral blood separation method, comprising:
adding a peripheral blood sample liquid in a sample bag and a gradient liquid into a centrifuge cup;
collecting a target cell liquid into an intermediate bag after centrifugation of the peripheral blood sample liquid and the gradient liquid in the centrifuge cup;
adding the target cell liquid in the intermediate bag and a washing liquid to the centrifuge cup for washing and replacement to obtain a pre-product; and
mixing the pre-product and a cell dilution liquid to obtain a final product.

2. The fully automatic peripheral blood separation method of claim 1, further comprising:
before adding the peripheral blood sample liquid, degassing the peripheral blood sample liquid in the sample bag,
wherein the peripheral blood sample liquid is added to the centrifuge cup at a liquid feeding rate of 18 to 22 mL/min.

3. The fully automatic peripheral blood separation method of claim 1, wherein different layers are formed in the centrifuge cup following the centrifugation, and
wherein the fully automatic peripheral blood separation method further comprises:
after the centrifugation, backflowing the peripheral blood sample liquid remaining in a pipe to the sample bag.

4. The fully automatic peripheral blood separation method of claim 1, wherein a volume of the peripheral blood sample liquid is greater than a separation volume of the centrifuge cup, and
wherein the peripheral blood sample liquid is fed and separated for a plurality of times based on the volume of the peripheral blood sample fluid and the separation volume of the centrifuge cup.

5. The fully automatic peripheral blood separation method of claim 4, further comprising:
after collecting the target cell liquid, cleaning the centrifuge cup and a pipe connecting the centrifuge cup and the intermediate bag.

6. The fully automatic peripheral blood separation method of claim 1, wherein adding the target cell liquid in the intermediate bag and the washing liquid to the centrifuge cup comprises:
i) discharging the target cell liquid from the intermediate bag to the centrifuge cup; then
ii) adding the washing liquid to the intermediate bag; and then
iii) discharging the washing liquid to the centrifuge cup.

7. The fully automatic peripheral blood separation method of claim 1, wherein the washing and replacement are carried out for a specific number of times that is in a range from 1 to 3 times.

8. The fully automatic peripheral blood separation method of claim 1, further comprising:
discharging the final product into a product bag; and
cleaning the centrifuge cup with the cell dilution liquid and discharging a cleaned liquid into the product bag for a specific number of times that is in a range from 1 to 20 times.

9. The fully automatic peripheral blood separation method of claim 8, further comprising:
after cleaning the centrifuge cup, packaging the final product in the product bag into one or more individual bags.

10. A fully automatic peripheral blood separation device, comprising:
a plurality of components comprising at least one of:
a centrifuge configured to provide a rotational power to a centrifuge cup,
a spin valve configured to control opening or closing of a pipe coupled to the centrifuge cup,
a peristaltic pump configured to pump a liquid into or out of the centrifuge cup, or
one or more scales each configured to weigh an amount of a respective liquid, and
a controller coupled to one or more components of the plurality of components and configured to perform one or more operations for fully automatic peripheral blood separation, the one or more operations comprising:
controlling the spin valve to open the pipe and the peristaltic pump to pump a peripheral blood sample liquid and a gradient liquid into the centrifuge cup;
controlling the centrifuge to rotate to separate a target cell liquid from the peripheral blood sample liquid and the gradient liquid in the centrifuge cup;
controlling to collect the target cell liquid into an intermediate bag;
controlling to add the target cell liquid in the intermediate bag and a washing liquid to the centrifuge cup for washing and replacement to obtain a pre-product; and
controlling to mix the pre-product and a cell dilution liquid to obtain a final product.

11. A peripheral blood mononuclear cell (PBMC) separating and packaging device, the device comprising:
a washing liquid assembly;
a density gradient liquid assembly;
a sample liquid assembly;
a waste liquid assembly;
a preparation liquid assembly;
a semi-finished product liquid assembly;
a packaging assembly; and
a separation cup,
wherein the separation cup is connected to a first three-way joint via a cup inlet pipe, and the first three-way joint is further connected to a liquid inlet pipe and a collection pipe,
wherein the washing liquid assembly, the density gradient liquid assembly, the sample liquid assembly, and the waste liquid assembly are connected to the liquid inlet pipe via a first five-way joint, and
wherein the preparation liquid assembly, the semi-finished product liquid assembly, and the packaging assembly are connected to the collection pipe via a first four-way joint.

12. The PBMC separating and packaging device of claim 11, wherein the washing liquid assembly comprises a first washing liquid branch, a second washing liquid branch, and a washing liquid pipe, and
wherein one end of the washing liquid pipe is connected to the first five-way joint, and the other end of the washing liquid pipe is connected to the first washing liquid branch and the second washing liquid branch via a second three-way joint.

13. The PBMC separating and packaging device of claim 12, wherein a protective cap, a first piercer, a first Luer connector, and a first pipe clamp that are provided on the first washing liquid branch, and
wherein a second piercer and a second pipe clamp that are provided on the second washing liquid branch.

14. The PBMC separating and packaging device of claim 11, wherein the density gradient liquid assembly comprises:
a density gradient liquid pipe, and
a density gradient liquid bag,
wherein one end of the density gradient liquid pipe is connected to the first five-way joint, the other end of the density gradient liquid pipe is connected to the density gradient liquid bag,
wherein a third pipe clamp is provided on the density gradient liquid pipe, and
wherein a liquid filter and a fourth pipe clamp are provided on the density gradient liquid bag.

15. The PBMC separating and packaging device of claim 11, wherein the sample liquid assembly comprises a sample pipe connected to the first five-way joint, and
wherein a third piercer, a second Luer connector, a connecting pipe, a blood filter and a fourth pipe clamp are provided on the sample pipe.

16. The PBMC separating and packaging device of claim 11, wherein the waste liquid assembly comprises a waste liquid pipe, and
wherein one end of the waste liquid pipe is connected to the first five-way joint, the other end of the waste liquid pipe is connected to a waste liquid bag, and a fifth pipe clamp is provided on the waste liquid pipe.

17. The PBMC separating and packaging device of claim 11, wherein the preparation liquid assembly comprises:
a first preparation liquid branch,
a second preparation liquid branch, and
a preparation liquid pipe,
wherein one end of the preparation liquid pipe is connected to the first four-way joint, the other end of the preparation liquid pipe is connected to the first preparation liquid branch and the second preparation liquid branch via a fourth three-way joint, and
wherein a seventh pipe clamp and a fourth piercer are provided on the first preparation liquid branch, an eighth pipe clamp and a preparation liquid bag are provided on the second preparation liquid branch, and the preparation liquid bag is provided with a third Luer connector.

18. The PBMC separating and packaging device of claim 11, wherein the semi-finished product liquid assembly comprises a semi-finished product bag pipe, and
wherein one end of the semi-finished product bag pipe is connected to the first four-way joint, the other end of the semi-finished product bag pipe is connected to the semi-finished product bag, and a ninth pipe clamp is provided on the semi-finished product bag pipe.

19. The PBMC separating and packaging device of claim 11, wherein the packaging assembly comprises a packaging pipe, and
wherein one end of the packaging pipe is connected to the first four-way joint, the other end of the packaging pipe is connected to a cryopreservation container, and a tenth pipe clip is provided on the packaging pipe.

20. The PBMC separating and packaging device of claim 11, wherein the separation cup is connected to the cup inlet pipe and a pressure monitoring pipe via a third three-way joint, and a sixth pipe clamp and a pressure monitoring joint are provided on the pressure monitoring pipe.
